(19) United States
(12) Patent Application Publication
(10) Pub. No.: US 2012/0004205 A1
(43) Pub. Date: Jan. 5, 2012

(54) IL-13 INDUCED GENE SIGNATURE FOR EOSINOPHILIC ESOPHAGITIS

(75) Inventor: Marc Elliott Rothenberg, Cincinnati, OH (US)

(73) Assignee: Cincinnati Children's Hospital Medical Center, Cincinnati, OH (US)

(21) Appl. No.: 12/628,992

(22) Filed: Dec. 1, 2009

Related U.S. Application Data
(60) Provisional application No. 61/118,981, filed on Dec. 1, 2008.
(60) Provisional application No. 61/118,985, filed on Dec. 1, 2008.

(30) Foreign Application Priority Data
Dec. 1, 2009 (US) PCT/US09/66282

(51) Int. Cl. A61K 31/56; C40B 30/04; A61P 1/00; G01N 33/566; A61P 29/00; C07H 21/00; C12Q 1/68

(52) U.S. Cl. 514/180; 536/24.31; 506/9; 435/6.12; 435/7.92

(57) ABSTRACT

The present invention concerns methods useful in diagnosing, identifying and monitoring the progression of eosinophilic esophagitis through measurements of gene products induced by IL-13.

### CROSS REFERENCE TO RELATED APPLICATIONS

This application claims the benefit of priority under 35 U.S.C. §119(e) to U.S. Provisional Application Ser. No. 61/118,981, filed on Dec. 1, 2008, entitled IL-13 INDUCED GENE SIGNATURE FOR EOSINOPHILIC ESOPHAGITIS, U.S. Provisional Application Ser. No. 61/118,985, filed on Dec. 1, 2008, entitled METHODS OF DETERMINING EFFICACY OF GLUCOCORTICOID TREATMENT OF EOSINOPHILIC ESOPHAGITIS, and PCT Application Ser. No. PCT/US09/66282, entitled METHODS OF DETERMINING EFFICACY OF GLUCOCORTICOID TREATMENT OF EOSINOPHILIC ESOPHAGITIS, filed on Dec. 1, 2009, each of which is incorporated herein by reference in its entirety.

### GOVERNMENT RIGHTS

This subject matter disclosed herein was made with U.S. Government support under NIH Research Project Grant program NIH 1 U19 AI070235, and NIH Research Project Grant program NIH 5 R01 AI045898. The U.S. Government may have certain rights in the subject matter hereof.

### FIELD OF THE SUBJECT MATTER

The present field of the subject matter relates to a method for diagnosis and treatment of eosinophilic esophagitis. More specifically, the present subject matter relates to the pathogenesis of eosinophilic esophagitis as mediated by an IL-13 stimulated keratinocyte-derived transcriptome, and associated methods for treatment.

### BACKGROUND

All publications herein are incorporated by reference to the same extent as if each individual publication or patent application was specifically and individually indicated to be incorporated by reference. The following description includes information that can be useful in understanding the present subject matter. It is not an admission that any of the information provided herein is prior art or relevant to the presently claimed subject matter, or that any publication specifically or implicitly referenced is prior art.

Eosinophilic esophagitis (EE) is an emerging worldwide disease that mimics gastroesophageal reflux disease (GERD) and can lead to esophageal narrowing and stricture. Orenstein, S. R. et al., (2000) Am. J. Gastroenterol. 95: 1422-1430; Walsh, S. V. et al., (1999) J. Surg. Pathol. 23: 390-396; Liacouras, C. A. and Ruchelli, E. (2004) Curr. Opin. Pediat. 560-566; Vasilopoulus, S. et al., (2002) Gastrointest Endosc. 55: 99-106; Sant'Anna, A. M. et al., (2004) J. Pediatr. Gastroenterol. Nutr. 39: 373-377. Symptoms of EE include nausea, vomiting, abdominal pain, chest pain, heartburn, regurgitation, dysphagia, food impaction, poor appetite, early satiety, fussiness, and poor weight gain. EE is differentiated from GERD by the lack of response to acid suppression, the magnitude of mucosal eosinophilia and epithelial thickening, its male predominance and a high rate of association with atopy. Orenstein, S. R. et al., (2000) Am. J. Gastroenterol. 95: 1422-1430; Noel, R. J. et al., (2004) N.E. Med. 351: 940-941.

The current treatment of EE is with swallowed glucocorticoids such as fluticasone propionate (FP) and beclothemasone, man-made steroids that are related to naturally occurring steroid hormone, cortisol or hydrocortisone, produced by the adrenal glands. However, only a subset of EE patients experience remission from the disease following treatment with topical FP. This subset of patients respond to treatment with FP by exhibiting a decrease in esophageal eosinophils, epithelial hyperplasia, and symptom improvement.

T helper type 2 (Th2) cells are thought to induce allergic disorders through the secretion of an array of cytokines (e.g. IL-4, IL-5, IL-9, IL-13) that activate inflammatory and effector pathways both directly and indirectly. Ray, A. and Cohn, L., (1999) J. Clin. Invest. 104: 985-993; Kuperman, D. A. et al., (2002) Nat. Med. 8: 885-889. In particular, IL-4 and IL-13 are produced at elevated levels in allergic tissue and are thought to be central regulators of many of the hallmark features of eosinophilic disease. Hershey, G. K., (2003) J. Allergy Clin. Immunol. 111, 677-690, quiz 691. In addition to Th2 cells, inflammatory cells (for example eosinophils, basophils, and mast cells) within allergic tissue also produce IL-4 and IL-13. Straumann, A. et al., (2001) J. Allergy Clin. Immunol. 108: 954-961; Schmid-Grendelmeier, P. et al., (2002) J. Immunol. 169: 1021-1027. Whether these principles apply to the esophagus has not been determined. The production of IL-13 by esophageal cells including infiltrating cells has not been examined. The mechanisms by which IL-13 mediates its effects vary between tissues. Hershey, G. K., (2003) J. Allergy Clin. Immunol. 111, 677-690, quiz 691; Shim, Y. M. et al., (2006) J. Immunol. 177: 1918-1924; Ingram, J. L. et al., (2006) J. Immunol. 177: 4141-4148 as IL-13 induces dramatically different transcriptional profiles in different cell types. Lee, J. H. et al., (2001) Am. J. Respir. Cell Mol. Biol. 25:474-485; Horner, R. J. et al., (2006) Am. J. Phsiol. Lung Cell Mol. Physiol. 291: L502-511; Zheng, T. et al., (2003) J. Allergy Clin. Immunol. 111: 720-728; it is therefore important and significant to the present subject matter to determine which mechanism is operational in the esophagus.

Reports have shown that there are at least three different receptors that bind IL-4 and/or IL-13. IL-4 receptor type I, is composed of two chains, the IL-4Rα and gamma common chains. The Type II receptor, composed of IL-13α and IL-4Rα, can signal in response to IL-4 and IL-13. The IL-13Rα2 chain is expressed in various tissues, and exists in membrane-associated, cytoplasmic, and soluble (sIL-13Rα2) forms. Donaldson, D. D. et al., (1998) J. Immuno. 161: 2317-2124; Mentik-Kane, M. M. et al., (2004) Proc. Nat'l Acad. Sci. USA 101: 586-590, Mentik-Kane, M. M. and Wynn, T. A., (2004) Immunol. Rev. 202: 191-202, Zhang et al., (1997) J. Biol. Chem. 272: 9474-9480. IL-13Ra2 exhibits inducible expression vivo that is IL-13, and Signal Transducer and Activator of Transcription 6 (STAT6)-dependent. Wood, N. et al., (2003) J. Exp. Med. 197: 703-709; Aceves, S. S. et al., (2007) J. Allergy Clin. Immunol. 119: 206-212. IL-13-induced tissue remodeling including collagen deposition can be a key process in several diseases. Notably, esophageal biopsies from EE patients have evidence of extensive tissue remodeling including collagen deposition and angiogenesis, processes that have been shown to be induced by IL-13 in the lung. Aceves, S. S. et al., (2007) J. Allergy Clin. Immunol. 119: 206-212; Blackburn, M. R. et al., (2003) J. Clin. Invest. 112: 332-344.

Eotaxins are members of the cysteine-cysteine (C-C) chemokine family. Eotaxin-1 (CC chemokine ligand 11, CCL11) is chemotactic for eosinophiles, basophils, and type 2 helper T cells. Eotaxin-2 (CCL24) is identical with MPIF-2 (Myleoid Progenitor Inhibitory Factor-2) and is also known as Chemokine β-6. Eotaxin-3 (CCL26) is also known as IMAC, MIP-4α (Macrophage Inflammatory Protein-4 α), TSC-1 (Thymic Stroma Chemokine-1). All three eotaxins bind to the CCR3 G-protein coupled receptor, a member of the seven-membrane spanning receptor family.

IL-13 stimulates expression of eotaxin-1 and eotaxin-2 by a STAT6 dependent mechanism in the murine lung yet both chemokines have a different cellular expression even after IL-13 delivery to the lung. Zimmermann, N. et al., (2003) J. Allergy Clin. Immunol. 111: 227-242, quiz 243; Zimmermann, N. et al., (2000) J. Immunol. 165: 5839-5846; Pope, S. M. et al., (2005) J. Biol. Chem. 280: 13952-13961. All three human eotaxin promoters contain STAT6 consensus binding motifs, but their relative importance and mode of action differs. Blanchard, C. et al., (2005) Int. J. Biochem. Cell Biol. 37: 2559-2573; Hebenstreit, D. et al., (2005) Mol. Immunol. 42: 295-303; Hoeck, J. and Woisetschlager, M., (2001) J. Immunol. 167: 3216-3222; Hoeck, J. and Woisetschlager, M., (2001) J. Immunol. 166: 4507-4515; Matsukura, S. et al., (2001) Am. J. Respir. Cell Mol. Biol. 24:755-761. Interestingly, EE patients have an esophageal transcriptome characterized by 574 genes differentially expressed that is remarkably conserved between individuals despite their atopic status or gender. Blanchard, C. et al., (2006) J. Clin. Invest. 116: 536-547. Notably, eotaxin-3 is the most overexpressed gene within the EE transcriptome (53-fold increase compared with normal individuals). Interestingly, eotaxin-1 and eotaxin-2 are not significantly induced in EE patients. Together with the localization of eotaxin-3 to esophageal keratinocytes, these results support the involvement of keratinocyte-derived eotaxin-3 in disease pathogenesis. Blanchard, C. et al., (2006) J. Clin. Invest. 116: 536-547.

Thus, there is a need in the art for a better understanding of the molecular mechanisms and involvement of keratinocyte-derived eotaxin-3 in disease pathogenesis, and treatment methods for EE.

### SUMMARY

Embodiments of the invention relate to a method for diagnosis of eosinophilic esophagitis (EE), wherein the method of diagnosis includes: determining a level of Interleukin 13 (IL-13) expression; and prognosing a responsive case of eosinophilic esophagitis based upon the level of IL-13 expression.

Some embodiments of the invention can further include determining an expression level of at least one of, for example, Interleukin-4, Eotaxin-1, Eotaxin-2 and the like, along with determining a level of IL-13 expression, so as to diagnose EE.

In some embodiments of the invention, a method of diagnosing an EE subtype includes: determining the level of at least one glucocorticoid-responsive transcript; and diagnosing the EE subtype based upon the level of the transcript or transcripts. In some embodiments of the invention, the EE subtype is responsive to fluticasone propionate (FP) treatment In some embodiments of the invention, the glucocorticoid-responsive transcript includes the expression of a gene, for example, described in FIG. 6(D) herein and the like.

In some embodiments of the invention, a method of diagnosing an EE subtype includes: determining the level of at least one EE transcriptome gene that is not a glucocorticoid-responsive transcript; and diagnosing the EE subtype based upon the level of the transcript or transcripts. In some embodiments of the invention, the glucocorticoid is, for example, fluticasone propionate and the like.

In some embodiments of the invention, a method of treating EE in an individual includes: determining the presence of a glucocorticoid-responsive gene expression profile; and treating the individual based upon the profile. In some embodiments of the invention, the glucocorticoid-responsive gene profile includes the expression of IL-13. In some embodiments of the invention, the IL-13 is expressed in, for example, peripheral blood mononuclear cells and/or esophageal epithelial cells and the like.

In some embodiments of the invention, a kit for the detection of a level of one or more genes associated with EE is disclosed, wherein the kit can include: complementary oligonucleotide probes, for example, and the like, to subsequences of the one or more genes. In some embodiments of the invention, the kit can include probes, wherein the probes can be used in one or more of, for example, a gene chip, a PCR protocol, and the like.

In some embodiments of the invention, a method of determining the effectiveness of a treatment for EE, includes: administering the treatment to a cell, tissue, or individual; and analyzing the cell, tissue, or individual for the presence or absence of at least one of, for example, an IL-13 response, and the like, and elevated expression of at least one, for example, EE transcriptome gene, and the like.

In some embodiments of the invention, a method for determining whether a reflux patient is an EE patient is disclosed and includes: analyzing a sample from the patient to determine a profile of a EE transcriptome expression, wherein the EE transcriptome is indicative of an EE condition.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the IL-13 receptor chain expression in esophageal cells and eotaxin-3 production by esophageal cell lines following IL-13 stimulation. (A) The TE-1, TE-6, TE-7, and TE-13 esophageal cell lines were subjected to PCR analysis for IL-4Ra, IL-13Ra1, IL-13Ra2, and glyceraldehyde-3-phosphate dehydrogenase (GAPDH) mRNA expression. (B) Flow cytometric analysis of IL-4R, IL-13Ra1, and IL-13Ra2 chain expression in the TE-7 cell line (dark lines). Controls were performed with an irrelevant IgG1 (gray lines). (C) The TE-1, TE-6, TE-7, and TE-13 esophageal cell lines were stimulated for 24 hours with IL-13 (0, 1, 10, and 100 μg/mL). The fold increase of eotaxin-3 mRNA expression compared with that of the untreated cells is shown. (D & E) TE-7 cells were cultured with IL-13 (0, 1, 10, and 100 ng/mL) for 1, 6, 12, 24, and 48 hours. Eotaxin-3 protein released in the culture supernatant was quantified by means of ELISA. Results are presented as means 6 range and are representative of at least 3 experiments performed in triplicate

FIG. 2 depicts the rapid amplification of cDNA ends (RACE) of the eotaxin-3 gene. (A) Schematic representation of the genomic structure of the eotaxin-3 gene showing the position of the two consensus STAT6 responsive elements (STAT6 RE) and the putative upstream exon 1. (B & C) Esophageal cell line (TE-13) and eosinophilic esophagitis (EE) patient RNA (EE1, EE2, EE3) were subjected to 5′ or 3′ RACE and sequenced. Starting codon (ATG) and polyadenylation signal (AATAAA) are notated in bold. Alignments include GeneBank mRNA sequences and genomic sequences.

FIG. 3 depicts human eotaxin-3 promoter activity after IL-13 stimulation and the role of STAT6. (A) TE-7 cells were transfected with pGL3-Basic (Promega, Madison, Wis.) containing the eotaxin-3 promoter (P800) and phRLTK, a plasmid that bears the Renilla luciferase gene and can be used to monitor the efficiency of transfection (Promega, Madison, Wis.). Renilla luciferase encoded by phRLTK was used as an internal control for firefly luciferase normalization. Cells were stimulated with IL-13 (0, 1, 10, and 100 ng/mL). (B) TE-7 cells were transfected with pGL3-Basic containing different lengths of the eotaxin-3 promoter (P800, P500, and P100) and promoters containing mutations in the STAT6-responsive elements (MUT1, MUT2, and MUT1&2). (C) The TE-7 cells were cotransfected with P800 and a dominant negative form of STAT6 (DNSTAT6) or the empty vector (EV). (D) TE-7 cells were cotransfected with P800 and the expression vector containing STAT6:ER. The cells were stimulated with 4-hydroxytamoxifene (4HT; 10 mmol/L). Results are presented as the ratio of the luciferase firefly/Renilla activities. (E) Esophageal keratinocytes (TE-7) were pretreated with IL-13 (0 or 100 μg/mL) and actinomycin D (Actino; 0 or 10 mmol/L) for 0 to 48 hours. Results are presented as a percentage of eotaxin-3 mRNA compared with time in hours (100%; black and gray dashed lines for media and IL-13, respectively). Trend lines (black and gray lines for media and IL-13, respectively) were calculated

FIG. 4 depicts gene expression analysis in primary esophageal cells after IL-13 stimulation and comparison with the EE transcript signature. (A) The 54,765 genes of the Human Genome U133 Set (HG-U133) Affymetrix Genechip® were subjected to fold-change filter in patients with EE versus healthy subjects and IL-13-stimulated primary cell cultures versus unstimulated cells. Spearman correlation and linear regression were calculated. (B) The list displays 33 transcripts that were upregulated 5-fold or greater and 5 transcripts that were downregulated 4-fold or greater compared with unstimulated cells. (C) The genes modified by 1.5-fold or greater on average in IL-13 stimulated cells (100 μg/mL) are presented in a heat diagram in 3 primary-culture patient biopsy specimens (1, 2, and 3), unstimulated and stimulated. Upregulated genes are shown in red, and downregulated genes are shown in blue. The magnitude of the gene changes is proportional to the darkness of the color. (D) The fold increase of eotaxin-3 mRNA expression compared with the untreated value was quantified by means of real-time PCR. (E) Eotaxin-3 released in the culture supernatant is expressed in nanograms per milliliter. Results are presented as means 6 range and are representative of experiments performed in 5 different patients.

FIG. 5 depicts the IL-13 and IL-4 mRNA expression in biopsy samples from healthy (NL) subjects and patients with EE. The expression of IL-13 (A) and IL-4 (B) is shown. Each mRNA value is normalized to glyceraldehyde-3-phosphate dehydrogenase (GAPDH) mRNA expression from the same sample and is expressed as a fold increase. The black lines represent the mean value in each group. P values were calculated by using the Mann-Whitney U test (2 groups; n 5 8-9 and 13-21 subjects for the healthy and EE groups, respectively).

FIG. 6 depicts the effect of glucocorticoids on the EE transcriptome and resistant genes. (A & B) The expression of IL-13 (A) and eotaxin-3 (B) mRNA is shown in healthy subjects (NL), patients with EE, and patients with EE treated successfully with FP (n 5 8-9, 13-19, and 6-8 subjects for the NL, EE, and FP groups, respectively). (C) Total mRNA was subjected to microarray analysis. Upregulated genes are shown in red, and down-regulated genes are shown in blue. Each column represents a separate patient (NL, EE, and FP Rx), and each line represents a gene. (D) Genes that are resistant to glucocorticoid therapy are shown with their Affymetrix accession numbers and their fold change in patients with EE and in treated patients with EE. (E) Expression of cadherin-26 (CDH26) was quantified by means of real-time PCR. Each data point corresponds to a separate individual (n 5 9, 11, and 7 subjects for the NL, EE, and FP groups, respectively). P values were calculated using Kruskal-Wallis tests (3 groups).

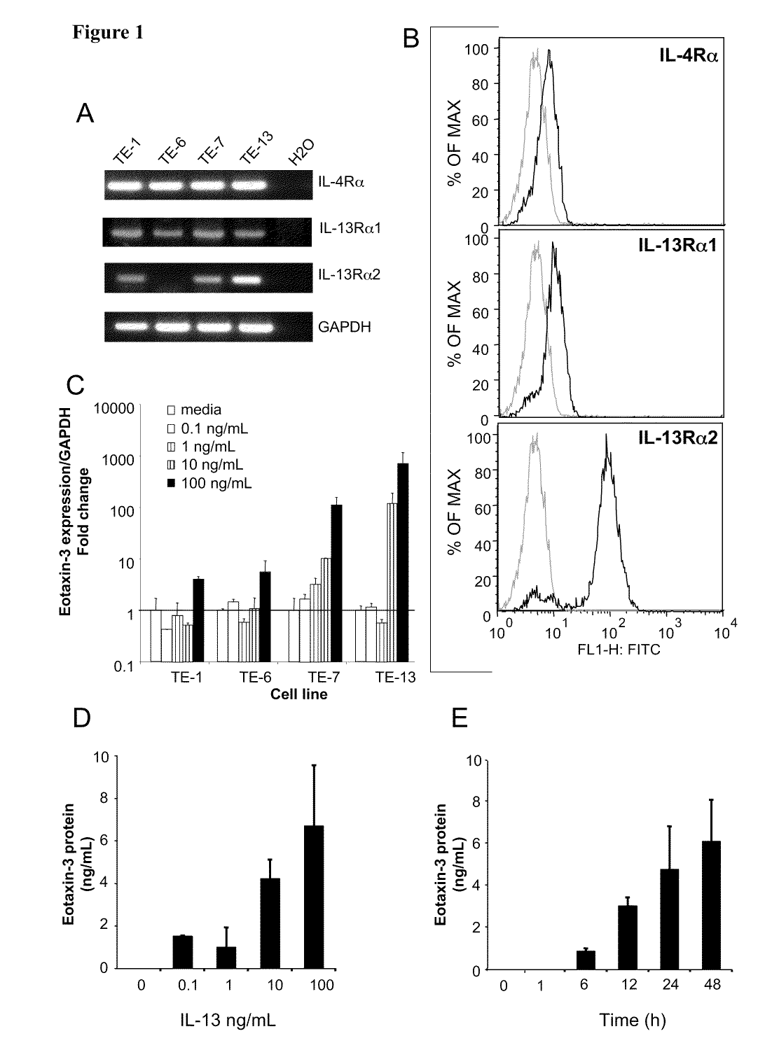

### DETAILED DESCRIPTION OF THE INVENTION

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

As used herein, the term “level” includes a gage of, or measure of the amount of, or concentration of a transcription product, for instance mRNA, or a translation product, for instance a protein or polypeptide. A level of RNA expression can be expressed in units such as transcripts per cell or nanograms per microgram of tissue. A level of a polypeptide can also be expressed as nanograms per microgram of tissue or nanograms per milliliter of a culture medium, for example. Alternatively, relative units can be employed to describe an expression level. For example, when an assay has an internal control, for instance a control gene, for example glyceraldehyde 3-phosphate dehydrogenase (GAPDH), for which the expression level is either known or can be accurately determined, unknown expression levels of other genes can be compared to the known internal control.

Once an expression level is determined for a gene, a profile can be created. As used herein, the term “profile,” for example a gene expression profile, refers to a repository of the expression level data that can be used to compare the expression levels of different genes, in whatever units are chosen. The term “profile” is also intended to encompass manipulations of the expression level data derived from a cell, tissue or individual. For example, once relative expression levels are determined for a given set of genes, the relative expression levels for that cell, tissue or individual can be compared to a standard to determine if expression levels are higher or lower relative to the same genes in a standard. Standards can include any data deemed by one of skill to be relevant for comparison. A standard can be prepared by determining the average expression level of a gene in a normal population, a normal population being defined as subjects that do not have EE. A standard can also be prepared by determining the average expression level of a gene in a population of individuals with EE.

As used herein, the term “determining,” and grammatical derivatives thereof, such as, but not limited to “determine,” or “determined,” can include measuring the expression level, for example, the amount or concentration of a nucleic acid or protein marker of the invention. The term thus can refer to use of materials, compositions and methods of the embodiments of the invention for qualitative and quantitative assessment. A qualitative determination of the level of a marker can include comparing the level of a marker in a sample with the level of the marker in a control sample or with the level of the marker obtained from the same patient but at a different time point. A quantitative determination includes measuring the amount or concentration of the level of a nucleic acid or protein that is encoded by or that corresponds to the particular marker. For example, detecting a change in expression levels can include quantifying a change of any value between 10% and 90%, or of any value between 20% and 80%, 30% and 70%, 40% and 60% or over 100%, of a marker of the invention relative to a control. Detecting an increase in gene expression levels can include quantifying a change of any value between 1.5 fold to 10000 fold or more of any of the markers of the invention relative to a control. More particularly, an increase in gene expression levels can include changes in value of 2, 5, 10, 25, 50, 100, 1000 fold or more.

As used herein, the term “detect” and all other forms of the root word “detect” can refer to the ascertainment of the presence or absence of one or more markers, quantization of one or more targets, or assessment of the presence or absence of a threshold value of one or more markers. A threshold value can be determined experimentally, empirically, or theoretically. A threshold value can also be arbitrarily selected.

As used herein, the term “gene chip” refers to a matrix, the basic material of which is, for example, glass or nylon, onto which DNA fragments are immobilized, it being possible for the application of the DNA to be carried out for example by (a) a photolithographic process (DNA is synthesized directly on the array matrix), (b) a microspotting process (externally synthesized oligonucleotides or PCR products are applied to the matrix and covalently bonded thereto), or (c) by a microspraying process (externally synthesized oligonucleotides or PCR products are sprayed onto the matrix without contacting by an ink-jet printer) (cf. R. Rauhut, Bioinformatik (Bioinformatics), pp 197-199, ed: Wiley-VCH Verlag GmbH, Weinheim, 2001). A gene chip that represents genomic sequences of an organism is typically referred to as a genomic DNA gene chip. The analysis of the measured values obtained with the aid of a gene chip is gene chip analysis.

As used herein, the term “transcript” can refer to an RNA molecule that is derived through the process of transcription from DNA. Transcripts can also be represented in some situations by proteins translated from RNA transcripts. A “glucocorticoid” is a steroid hormone capable of binding to the glucocorticoid receptor. A “glucocorticoid-responsive transcript” refers to an RNA molecule or molecules whose expression is either increased or decreased, by 1.5, 2, 5, 10, 25, 50, 100, 1000, 10000 fold or more, in the presence of a glucocorticoid.

As used herein, the term “subsequence” refers to any part of a polynucleotide sequence that is less than the entire polynucleotide sequence, and that would be suitable to perform the method of analysis. A person skilled in the art can choose the position and length of a subsequence by applying routine experiments. For example, a subsequence of a polynucleotide can be any contiguous sequence of at least about 10, about 25, about 50, about 100, about 200, about 300, about 400, about 800, or about 1,000 nucleotides, or more.

As used herein, the term “treating” or “treatment,” with respect to disease encompasses (1) preventing the disease, for example, causing the clinical symptoms of the disease not to develop in an animal that is exposed to or predisposed to the disease but does not yet experience or display symptoms of the disease, (2) inhibiting the disease, for example, arresting the development of the disease or its clinical symptoms, or (3) relieving the disease, completely or partially, for example, causing regression of the disease or its clinical symptoms. It will be appreciated by those skilled in the art that treatment extends to prophylaxis as well as the treatment of inflammation or other symptoms.

As used herein, the term “presence” refers to when a molecule can be detected using a particular detection methodology. Also as used herein, the term “absence” refers to when a molecule cannot by detected using a particular detection methodology.

As used herein, the term “elevated” encompasses activity that is increased above the level found typically in cells or tissue from an individual free of EE relative to the same type of cell or tissue from an individual diagnosed with EE. Generally, elevated activity is at least about 1.5, 2, 5, 10, 25, 50, 100, 1000, 10000 fold, or more greater than that in corresponding cells or tissues from an individual free of EE.

As used herein, the term “condition” includes any pathological or non-pathological syndrome, sign, symptom or physiological event from which a change is desired or beneficial to a mammal.

As used herein, the term “administering” and grammatical derivatives thereof, refers to, in the most general sense, to the contacting of a compound, reagent, or material directly to a cell or tissue or to the environment that surrounds the cell or tissue. The term “administer” also encompasses any route of introducing or delivering to an individual a compound, reagent, or material to perform its intended function. Administration can be carried out by any suitable route, including, but is not limited to, topical, transdermal, intranasal, vaginal, rectal, oral, subcutaneous intravenous, intra-arterial, intramuscular, intraosseous, intraperitoneal, epidural and intrathecal.

As used herein, the term “patient” encompasses an individual with symptoms of and or suspected of having EE. Patient includes human beings, but can also include animals generally. Patients can be female or male and person(s) of all ages.

The term “oligonucleotide” refers to a relatively short polynucleotide, typically less than or equal to 150 nucleotides long, for example, between 5 and 150 nucleotides in length, preferably between 10 and 100 nucleotides in length, or more preferably between 15 and 50 nucleotides in length. As used herein, the term “oligonucleotide” can encompass longer or shorter polynucleotide chains. An “oligonucleotide” can hybridize to other polynucleotides or target nucleic acids, therefore serving as a probe for polynucleotide detection. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “complementary” refers to the concept of sequence complementarity between regions of two polynucleotide strands. It is known that an adenine base of a first polynucleotide region is capable of forming specific hydrogen bonds (“base pairing”) with a base of a second polynucleotide region that is antiparallel to the first region if the base is thymine or uracil. Similarly, it is known that a cytosine base of a first polynucleotide strand is capable of base pairing with a base of a second polynucleotide strand that is antiparallel to the first strand if the base is guanine. A first region of a polynucleotide is complementary to a second region a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” can refer to a first polynucleotide that is 100% or “fully” complementary to a second polynucleotide and thus forms a base pair at every nucleotide position. “Complementary” also can refer to a first polynucleotide that is not 100% complementary (e.g., 90%, 80%, 70% complementary or less) contains mismatched nucleotides at one or more nucleotide positions.

As used herein, the term “probe” encompasses a polymer (e.g. a DNA, RNA, PNA, LNA chimera, linked polymer as well as combinations thereof (for example, an LNA/DNA chimera)) designed to sequence specifically hybridize to a target sequence of interest. An “oligonucleotide probe” refers to a nucleic acid probe, of either DNA or RNA, used to detect the presence of a complementary target sequence by hybridization with the target sequence.

As used herein, the term “prognosis” means a prediction of the probable outcome and/or course of a disease, it can be measured by reference to any suitable parameter recognized by those of skill in the art.

As used herein, the term “sample” refers to a biological material that is isolated from its natural environment and contains a polynucleotide. A “sample” according to the invention can include a purified or isolated polynucleotide, or it can include a biological sample such as a tissue sample, a biological fluid sample, or a cell sample including a polynucleotide. A biological fluid can be, for example, blood, plasma, sputum, urine, cerebrospinal fluid, lavages, biopsy, for example esophageal biopsy or esophageal mucosal biopsy, and leukophoresis samples. Useful samples can be obtained from different sources, including, for example, but not limited to, from different individuals, different developmental stages of the same or different individuals, different diseased individuals, normal individuals, different disease stages of the same or different individuals, individuals subjected to different disease treatments, individuals subjected to different environmental factors, individuals with predisposition to a pathology, individuals with exposure to an infectious disease. Useful samples can also be obtained from in vitro cultured tissues, cells, or other polynucleotide containing sources. The cultured samples can be taken from sources including, but are not limited to, cultures (for example, tissue or cells) cultured in different media and conditions (for example, pH, pressure, or temperature), cultures (for example, tissue or cells) cultured for different period of length, cultures (for example, tissue or cells) treated with different factors or reagents (for example, a drug candidate, or a modulator), or cultures of different types of tissue or cells.

Embodiments of the invention are also directed to a kit for the detection of expression levels of one or more genes, and may include an array of immobilized oligonucleotide probes complementary to subsequences of said one or more genes. Likewise, the kit can include materials for detection of genes; gene expression; expression, accumulation, and/or localization of proteins; and the like, including, for example, reagents, equipment, and/or instrumentation for ELISA, gene-chip expression analysis, RT-PCR, and the like. The kit is an assemblage of materials or components, including at least one of the inventive compositions. Thus, in some embodiments the kit contains a composition including polynucleotides encoding glucocorticoid-responsive transcripts, as described above.

The exact nature of the components configured in the inventive kit depends on its intended purpose. For example, some embodiments are configured for the purpose of detecting an expression profile of glucocorticoid-regulated genes.

Instructions for use can be included in the kit. “Instructions for use” typically include a tangible expression describing the technique to be employed in using the components of the kit to affect a desired outcome, such as to prepare a gene array for the diagnosis and/or prognosis of efficacy of glucocorticoid treatment of eosinophilic esophagitis. Optionally, the kit also contains other useful components, such as, diluents, buffers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in preparing a nanoconjugate. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing a solution of polynucleotides encoding the IL-13 or eotaxin-3 transcript. The packaging material generally has an external label that indicates the contents and/or purpose of the kit and/or its components.

One skilled in the art, having the benefit of this description of the invention, will recognize many methods and materials similar or equivalent to those described herein that can be used in the practice of the embodiments of the invention. Indeed, the embodiments of the invention is in no way limited to the exemplary methods and materials described herein. One skilled in the art will recognize many methods and materials similar or equivalent to those described herein that can be used in the practice of the present subject matter. Indeed, the present subject matter is in no way limited to the methods and materials described.

“EE” as used herein is an abbreviation for Eosinophilic esophagitis.

“ER” as used herein is an abbreviation for Estrogen receptor.

“FP” as used herein is an abbreviation for Fluticasone propionate. FP is a white to off-white powder, with the empirical formula C₂₅H₃₁F₃O₅S and a molecular weight of 550.6. Fluticasone refers to the synthetic, trifluorinated, corticosteroid having the chemical name of S-fluoromethyl-6α,9-difluoro-11β-hydroxy-16α-methyl-3-oxo androsta-1,4-diene-17β-carbothioate, 17-propionate, and salts and derivates thereof. Only a subset of EE patients experience remission from the disease following treatment with topical FP.

“STATE” as used herein is an abbreviation for Signal Transducer and Activator of Transcription 6.

The present subject matter addresses the molecular mechanisms involved in the development of EE by focusing on the signaling pathway responsible for the induction of the EE specific transcriptome. A transcriptome is a collection of RNA transcripts transcribed in a specific tissue, whether coding or non-coding, and preferably contains all or substantially all of the RNA transcripts generated in the tissue. These transcripts include messenger RNAs (mRNA), alternatively spliced mRNAs, ribosomal RNA (rRNA), transfer RNAs (tRNAs) in addition to a large range of other transcripts, which are not translated into protein such as small nuclear RNAs (snRNAs), antisense molecules such as short interfering RNA (siRNA) and microRNA and other RNA transcripts of unknown function. The transcriptome also includes proteins translated from the RNA transcripts within the transcriptome, which is an extension and reflection of gene transcription within the transcriptome. The EE specific transcriptome is the collection of RNA transcripts observed in individuals with EE.

Based on data implicating eotaxin-3 in disease pathogenesis, focus was placed on this particular gene. The present subject matter demonstrates that the expression of the eotaxin-3 gene was markedly upregulated by IL-13 (IL13, P600, ALRH, BHR1, MGC116786, MGC116788 or MGC116789) stimulation of immortalized as well as EE patient derived esophageal epithelial cells (keratinocytes). Translational studies revealed that IL-13, but not IL-4, mRNA level was increased 16-fold in esophageal biopsies from EE patients compared to normal individuals. Furthermore, IL-13 treatment of esophageal keratinocytes was sufficient to induce a genome-wide microarray transcript profile that overlapped (22%) with the EE transcriptome and included eotaxin-3 as the most highly induced gene. Notably, the present subject matter shows both the EE and IL-13-induced transcriptome to be largely reversible with glucocorticoid treatment in vivo. Taken together, it can be said that the pathogenesis of EE is mediated by an IL-13 and involving eotaxin-3, which stimulates the expression of a particular collection of RNA molecules that is largely reversible with corticosteroid treatment. In addition, an in vivo IL-13-associated gene signature is defined herein with potential value for efficacy assessment of anti-IL-13 therapeutics.

IL-13 was first recognized for its effects on B cells and monocytes, where it upregulated class II expression, promoted IgE class switching and inhibited inflammatory cytokine production. It was also thought to be functionally redundant with IL-4. However, studies conducted with knockout mice, neutralizing antibodies, and novel antagonists demonstrate that IL-13 possesses several unique effector functions that distinguish it from IL-4. Wynn (2003) Ann. Rev. Immunol. (21):425-456.

In the lung, IL-13 is the central mediator of allergic asthma, where it regulates eosinophilic inflammation, mucus secretion, and airway hyperresponsiveness. Also, IL-13 is a mediator of tissue fibrosis in asthma, which indicates that it is a key regulator of the extracellular matrix.

IL-4, IL-12, IL-18, IFN-y, IL-10, TGF-β, TNF-α, and the IL-4/IL-13 receptor complex play roles in regulating IL-13 production and/or function.

Accordingly, the present subject matter demonstrates that: 1) epithelial cell lines of the esophagus express the IL-13R1 and IL-13R2 polypeptides; 2) IL-13 specifically induces eotaxin-3 (but not eotaxin-1 or -2) expression in esophageal epithelial cell lines; 3) IL-13 induced eotaxin-3 expression is dependent upon the transcription factor STAT6 via a proximal promoter binding element at by position -89; 4) IL-13 induces eotaxin-3 overexpression in primary esophageal keratinocytes from EE patient biopsies; 5) IL-13 induces an EE-like transcriptome in primary keratinocyte cultures that have considerable overlap with the human EE transcriptome; 6) IL-13, but not IL-4, mRNA is markedly upregulated in EE patient biopsies; and 7) the EE transcriptome, including the overexpression of IL-13 and eotaxin-3, is glucocorticoid reversible. Taken together, these findings support a model that specifically implicates IL-13 in EE pathogenesis.

In a preferred embodiment, the present subject matter discloses a method for treatment of EE by identifying IL-13-induced pathways and genes as the fundamental processes in the cause and manifestations of EE, and disclosing therapeutic agents that interfere with IL-13 to facilitate disease treatment.

In one embodiment, the present subject matter provides a method for treatment of EE by application of glucocorticoid, wherein the pathogenesis of EE is mediated by an IL-13-stimulated keratinocyte-derived transcriptome.

In another embodiment, the present subject matter discloses a method for diagnosing EE based on the expression of IL-13, which induces eotaxin-3 overexpression in primary esophageal keratinocytes.

In another embodiment, the present subject matter discloses a method for diagnosis of EE relative to a healthy subject by determining the presence of IL-13 mRNA and IL-4 mRNA, where upregulation of IL-13 indicates positive for EE. IL-4 (Interleukin-4, IL4) is a cytokine that has multiple biological roles, including the stimulation of activated B cells, induction of T cell proliferation, and the differentiation of CD4′ T cells into T_{H}2 cells. The cDNA sequence coding for human IL-4 was first described by Yokota et al. (1986) Proc. Nat'l Acad. Sci, USA 83:5894-5898.

In yet another embodiment, the present subject matter discloses a method for diagnosis of EE by identifying an in vivo IL-13-induced transcriptome that has utility for target assessment after anti-IL-13 therapeutics.

The findings of the present subject matter are consistent with a recent study in a colonic epithelial cell line, but contrast with the literature describing IL-13-induced eotaxin-1 mRNA stabilization in airway epithelial cells; this suggests, without being limited to a particular theory or mode of action, that different mechanisms can be utilized for the regulation of distinct eotaxin family members in different cell types. Blanchard, C. et al., (2005) Int. J. Biochem. Cell Biol. 37: 2559-2573; Atasoy, U. et al. (2003) J. Immunol. 171: 4369-4378. It is interesting to note that the proximal STAT6 responsive element is necessary and sufficient for transcriptional eotaxin-3 induction in esophageal epithelial cells. It is notable that the EE transcriptome and IL-13-induced genes do not include eotaxin-1 and eotaxin-2 in spite of the presence of STAT6 binding sites in both of these genes. Taken together, these results suggest, without being bound to a particular theory or mode of action, that the regulation of eotaxin-3 occurs differently from the other eotaxins, and that keratinocytes utilize a regulatory pathway unique from other cells.

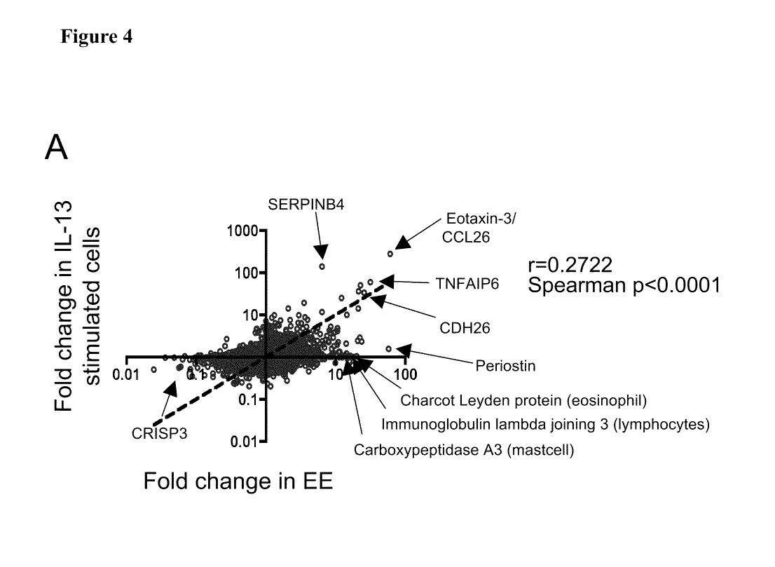

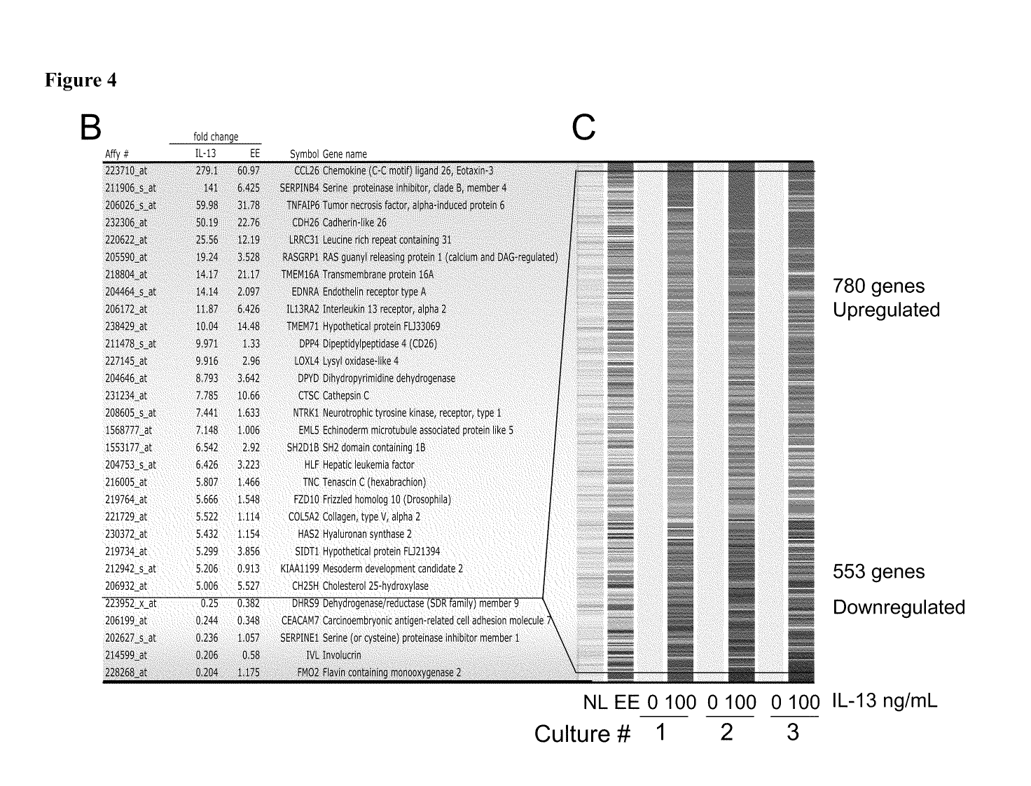

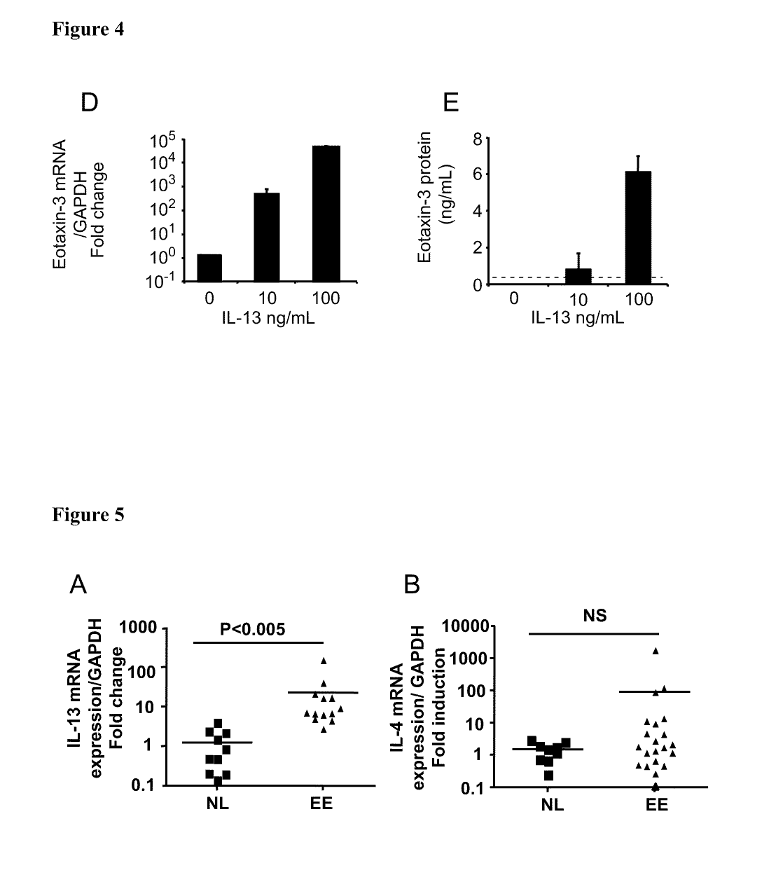

The EE transcriptome is 574 genes that were significantly modified in EE patients compared to normal biopsies, and the eotaxin-3 gene was the most upregulated gene (53-fold). A large number of EE-associated genes are directly induced by IL-13 in esophageal keratinocytes; therefore IL-13 can be a master regulator of the keratinocyte pathways involved in EE. In EE, the esophageal tissue undergoes changes marked by an abnormal accumulation of eosinophils, mast cells and lymphocytes epithelial cell hyperplasia, elongation of the papillae (endothelial cells and fibroblasts) and intensive lamina propria remodeling (likely involving fibroblasts). Liacouras, C. A. and Ruchelli, E. (2004) Curr. Opin. Pediat. 560-566; Sant'Anna, A. M. et al., (2004) J. Pediatr. Gastroenterol. Nutr. 39: 373-377; Straumann, A. et al., (2001) J. Allergy Clin. Immunol. 108: 954-961; Blackburn, M. R. et al., (2003) J. Clin. Invest. 112: 332-344; Blanchard, C. et al., (2005) Int. J. Biochem. Cell Biol. 37: 2559-2573; Kirsch, R. et al., (2007) J. Pediar. Gastroenterol. Nutr. 44:20-26; Furuta, G. J., (2002) Allergy Asthma Rep. 2:67-72; Dauer et al., (2005) Ann Otol. Rhinol. Laryngol. 114: 827-833; Parfitt, J. R. et al., (2006) Mod. Pathol. 19: 90-96; Blanchard, C. et al., (2006) J. Allergy Clin. Immunol. 1054-1059. In a minimalist model system, the stimulation of keratinocytes with IL-13 is able to partially reproduce the EE transcriptome indicating that this cell type largely accounts for the abnormal response seen in endoscopic biopsies. The striking overlap between the EE and the IL-13-induced transcriptomes (FIG. 4E), supports a model, without limitation to a particular theory or mode of action, in which IL-13-induced gene expression in keratinocytes makes an important contribution to the EE; not excluded are indirect or paracrine affects. Categorization of the modified genes into functional groups revealed that the upregulated and downregulated genes are involved in the control of cell division and epidermal differentiation, respectively. In a recent study, eotaxin-1 has been shown to increase skin keratinocyte proliferation, suggesting a possible autocrine pathway involving eotaxin-3 stimulation of CCR3 positive esophageal epithelial cells and a role for eotaxin in epithelial cell hyperplasia. The propensity of IL-13 to induce—directly or indirectly—the expression of proliferation markers and to decrease the expression of several keratinocyte differentiation markers is consistent with the IL-13/eotaxin-3/CCR3 axis as an attractive target for development of therapeutics that limit not only eosinophil chemoattraction but also epithelial cell proliferation in EE.

Using microarray analysis, IL-4 and IL-13 mRNA were neither detected nor upregulated in EE patients; however, several other Th2 inducible molecules were still detected Blanchard, C. et al., (2006) J. Clin. Invest. 116: 536-547. A highly sensitive method (real-time PCR), demonstrates that IL-13 mRNA is indeed overexpressed in EE patients (16-fold). This is consistent with previous studies showing an increase in IL-13 levels in stimulated peripheral blood mononuclear cells (PBMC) and eosinophils of EE patients. Straumann et al., (2005) Inflamm Bowel Dis. 11: 720-726; Yamazaki, K et al., (2006) Dig. Dis. Sci. 51: 1934-1941., PBMCs refers generally to any blood cell having a rounded nucleus. Mast cells, basophils, eosinophils, lymphocytes and smooth muscle cells are potent IL-13 producing cells. Straumann, A. et al., (2001) J. Allergy Clin. Immunol. 108: 954-961; Schmid-Grendelmeier, P. et al., (2002) J. Immunol. 169: 1021-1027; Yamazaki, K et al., (2006) Dig. Dis. Sci. 51: 1934-1941; however, the exact cellular source(s) of IL-13 in the esophageal biopsy are unknown. IL-4 was overexpressed in less than 50% of EE patients, therefore IL-13 is relatively more important in the mucosa for most EE patients. The increased production of IL-13 in EE is supported by the presence of known IL-4/13 induced genes, such as eotaxin-3, IL-13R α2; 15-lipoxygenase, and TNFAIP6. Hoeck, J. and Woisetschlager, M., (2001) J. Immunol. 167: 3216-3222; Chiaramonte, M. G. et al., (2003) J. Exp. Med. 197: 687-701; Wood, N. et al., (2003) J. Exp. Med. 197: 703-709.

Topical fluticasone propionate therapy has been shown to improve clinical symptoms, as well as endoscopic and microscopic features of EE. This study uncovers demonstrates that successful fluticasone propionate treatment reverses the molecular signature of EE. IL-13-induced eotaxin-3 expression is largely (but not completely) decreased by glucocorticoids treatment in vitro (data not shown). While topical glucocorticoids are known to have anti-inflammatory effects, they do not universally reduce all cytokines so it was important to determine the affect of topical fluticasone on the EE transcriptome. Notably, glucocorticoid treatment was associated with reduced IL-13 and eotaxin-3 production, indicating that the classic features of this Th2-associated pathway in the esophagus are largely reversible. These reversible genes include cell-specific transcripts from eosinophils, mast cells, lymphocytes, fibroblasts and epithelial cells, as well as chemoattractants, growth factors, and molecules involved in cell proliferation. These results are consistent with the decrease in eosinophils, mast cells, and epithelial hyperplasia following therapy. While the treated biopsies appear microscopically normal, the transcriptome still contains a small number of dysregulated genes. The residual expression of these genes in successfully treated EE patients implies that these genes can be part of the primary constitutive genetic defect inherent to the epithelium or have a reduced propensity to respond to glucocorticoid treatment. Although the exact function of these genes in the esophageal epithelium is not known, their role in other tissues implies that these genes might modify the elasticity, permeability or proliferation of the epithelium. Residual expression markers can have clinical value as they can serve as diagnostic criteria, irrespective of the degree of tissue inflammation (and the expression of the rest of the EE transcriptome). Additionally, these resistant genes can also help explain the chronic and relapsing nature of the disease.

In summary, the present subject matter provide new insight into the molecular pathogenesis of EE and associated methods for treatment of EE. The present subject matter discloses that disease pathogenesis involves a glucocorticoid reversible IL-13-induced keratinocyte transcriptome that includes eotaxin-3. These results underscore the value of new therapeutics that interfere with the IL-13/eotaxin-3/CCR3 axis; it can be that such agents would not only limit eosinophil accumulation, but also the keratinocyte proliferation characteristic of EE. Furthermore, the newly defined set of IL-13-induced EE transcripts are pertinent and valuable in the testing of clinical reagents that block IL-13 in patients. Accordingly, anti-IL-13 therapeutics hold significance for EE patients because of their clinical benefit, and also because the EE transcriptome provides a robust way to molecularly monitor drug efficacy and mechanism of action.

In addition, genes of the EE transcriptome can be useful in determining whether a patient is an EE patient or is suffering from some non-EE reflux condition/symptoms. For example, a clinician considering treatment of a patient with reflux symptoms, or consulting with a patient during or after a course of treatment, can make use of a gene chip or other means of analysis of EE transcripts, wherein presence of EE transcripts indicates that the patient suffers from EE and should be treated accordingly.

One skilled in the art will recognize many methods and materials similar or equivalent to those described herein, which could be used in the practice of embodiments of the invention. Indeed, the embodiments of the invention are in no way limited to the methods and materials described. For purposes of the embodiments of the invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

One skilled in the art can develop equivalent means or reactants without the exercise of inventive capacity and without departing from the scope of the invention.

### Example 1

### General

Esophageal keratin ocytes are IL-13 receptor positive and markedly produce eotaxin-3 following IL-13 stimulation by a transcriptional mechanism dependent upon STATE. Translational studies revealed that IL-13 mRNA level was markedly increased in esophageal biopsies from EE patients compared to normal individuals. Furthermore, IL-13 treatment of keratinocytes was sufficient to induce a global expression transcript profile that remarkably overlapped with the EE specific esophageal transcriptome. Lastly, the EE transcriptome was largely reversible with glucocorticoid treatment in vivo. The pathogenesis of EE is mediated by an IL-13-stimulated keratinocyte-derived transcriptome (involving eotaxin-3) that is largely reversible with corticosteroid treatment. Further, an in vivo IL-13 transcriptome is identified that can be used to assess anti-IL-13 therapeutics.

### Example 2

### Materials

Human esophageal adenocarcinoma cell line and squamous epithelial cells were provided by Dr Hainault (IARC, Lyon France). These cell lines, originally selected from esophageal tumors and well characterized by Nishihira et al. were maintained in RPMI medium (Invitrogen) supplemented with 10% FCS and 1% penicillin/streptomycin/amphotericin (Invitrogen). For primary culture, after informed consent was obtained and during routine endoscopy, one or two distal esophageal biopsies from EE patients were collected and subsequently digested with trypsin. These samples were cultured in modified F-media (3:1 F-12/DMEM) supplemented FBS 5%, with adenine (24.2 μg/mL), cholera toxin (10−4 μM), insulin (5 g/mL), hydrocortisone (0.4 μg/mL), and epidermal growth factor (10 ng/mL) in the presence of penicillin, streptomycin, and amphotericin (Invitrogen). Briefly, biopsies were digested with trypsin twice. Trypsin was neutralized in F-media without epidermal growth factor containing 1 to 5×10⁵ feeders (NIH 3T3 J2 cells irradiated 6000 rads). The following day, the media was changed with F-media containing epidermal growth factor (10 μg/mL) and changed every other day. Using these conditions and irradiated murine fibroblast NIH 3T3 G2 feeder cells, esophageal keratinocytes (E-cadherin positive) avidly grew and showed remarkable responsiveness to IL-13 stimulation ex vivo. EE patient-derived cells were cultured for 2 weeks and fibroblasts were depleted by differential trypsinization.

### Example 3

### Cytokine Treatment of Cells

For cytokine cell stimulation experiments, IL-13 (Preprotech) was added to the culture media of 70-80% confluent cells at doses from 0.1 to 100 μg/mL for 1 h to 72 h. The mRNA stability measurement was performed using actinomycin-D (10 g/mL) which was added to a culture media of cells 0.5 h before or 24 h after cytokine treatment. The cells were either treated with actinomycin-D in the presence or the absence of cytokine (10 μg/mL) for 1, 6, 12, 24, and 48 h. During the kinetic experiments, due to the short half-life of the actinomycin-D activity, media were changed every 6 hours.

### Example 4

### DNA Microarray Analysis

For each patient, one distal esophageal mucosal biopsy sample was immersed in RNA, later RNA stabilization reagent (Qiagen) and stored at 4 C. for <15 days. Total RNA was extracted using the RNAeasy Mini Kit (Qiagen) according to the manufacturer's recommendations. Hybridization to DNA microarray was performed by the MicroarrayCore at Cincinnati Children's Hospital Medical Center, as previously reported Blanchard, C. et al., (2006) J. Clin. Invest. 116: 536-547. The genome wide human Affymetrix U133A plus 2.0 Genechip® was used, and gene transcript levels were determined using algorithms in the Microarray Analysis Suite and GeneSpring software (Silicon Genetics) as previously described Blanchard, C. et al., (2006) J. Clin. Invest. 116: 536-547. For identification of the resistant genes, the EE transcriptome gene list was applied to fluticasone propionate treated patients and genes significantly expressed differently between FP-treated and NL patients were subjected to fold change filter (2). Response to therapy was defined as <1 eosinophils/high power field (hpf) and no epithelial cell hyperplasia as described herein.

### Example 5

### Ontology Assessment

The list of differentially expressed transcripts was subjected to gene ontology analysis using DAVID (database for annotation, visualization and integrated discovery) and EASE (expression analysis systematic explorer), a web-based (hyper text transfer protocol david<dot>abcc<dot>ncifcrf<dot>gov) application that allows access to a relational database of functional annotations.

### Example 6

### 3′ and 5′-Rapid Amplification of cDNA Ends

Total RNA (500 ng) was isolated from the TE-13 cell line and from human biopsies as previously reported. The SmartRACE™ cDNA Amplification kit (Clontech) was used for 3′RACE and 5′RACE following manufacturer's instructions. The gene-specific primers for eotaxin-3 were: 5′-CTTCCAATACAGCCACAAGCC CCTTCC-3′ for 3′RACE and 5′-GGAAGGGGCTTGTGGCTGTATTGGAAG-3′ for 5′RACE. The 3′RACE and 5′RACE products were subcloned into the pCR2.1 vector (Invitrogen) and sequenced by the DNA Core facility, University of Cincinnati.

### Example 7

### Reporter Constructs and Expression Vectors

Plasmids were constructed or obtained as described before Blanchard, C. et al., (2005) Int. J. Biochem Cell Biol. 37: 2559-2573. Briefly, plasmid denoted P800, P500 and P100 contained respectively the first 800 by 500 by or 100 by of the human eotaxin-3 gene promoter (pEO3) subcloned into KpnI/HindIII-digested pGL3-Basic (Promega) as previously described Blanchard, C. et al., (2005) Int. J. Biochem. Cell Biol. 37: 2559-2579. Directed mutagenesis of the eotaxin-3 promoter was performed using Quickchange Kit from Stratagene according to the manufacturer's instructions using 2 primer sets: (first set) 5′-CCCAACCACAAGAAggtaccGGAATTGTTTTCAGGGCCGTCTCAG-3′ and 5′-CTGAGACGGCCCTGAAAACAATTCC ggtacc TTCTGTGGTTGGG-3′ which include a KpnI restriction site in the -89 TTCtctcgGAA STAT6 consensus site and (second set) 5′-GTAACTTAGTTCAG ggatcc GGAATATTACTTAATTTCCTCTGC-3′ and 5′-GCAGAGGAAATTAAGTAATATTCC ggatcc CTGAACTAAGTTAC-3′ which include a KpnI restriction site in the -698 TTCactgGAA STAT6 consensus site. The STAT6:ER plasmid was constructed in the pCDNA3 plasmid as previously described Kamogawa, Y. et al., (1998) J. Immunol. 161:1074-1077.

The first 1989 bp of human STAT6 cDNA were amplified by sticky-end PCR (3 0 cycles) using the LA778 plasmid as a template. Primers used to obtain digested BamHI and EcoRI restriction sites were the BgIII fwd-1 gatctACCATGTCTCTGTGGGGTCTGGTC and fwd-2 tACCATGTCTCTGTGGGGTCTGGTC and the primers EcoRI rev-1 cTTCATGGGGTAGGAAGTGGTTG and rev-2 aattcTTCATGGGGTAGGAAGTGGTTG containing a chain termination codon. The 1989 bp long PCR product encoded the first 659 N-terminal-amino-acids of STAT6 corresponding to a dominant negative form. The PCR product was purified and subcloned with T4 DNA ligase into BgIII/EcoRI-digested pCEFL-HA vector. This plasmid is referred to DNSTAT628.

### Example 8

### Transient Transfections

Approximately 10⁴ TE-7 cells per well (24 well plate) were plated on day 1 and transfected by Lipofectamine™ reagent on day 2. Briefly, 500 μg of reporter plasmids were mixed with 3 μl of Lipofectamine™ reagent in 100 ml of serum-free RPMI medium. In all transfection experiments, a plasmid with a Renilla luciferase reporter gene under the control of a thymidine kinase promoter (pHRL-TK, 25 μg/well) was used as an internal control. In co-transfection experiments with DNSTAT6 or pCDNA3-STAT6:ER (ER-estrogen receptor) plasmids, the empty vectors pCEFL HA or pCDNA3 were added to each set of transfections, respectively, to ensure that each well received the same amount of DNA. Complete medium (0.5 mL) was added and cells were incubated for 48 h to 72 h. The transfection mixture was then replaced with fresh complete medium for an additional 12 h period before cytokine treatment. On day 4, cells were either left untreated or stimulated with cytokines (100 μg/mL) or 4-hydroxytamoxifene (4HT) (1 μM) for 24 h to 48 hours. Cells were then lysed and the Firefly and Renilla luciferase activities were measured in a luminometor (Microlite, Dynatec Laboratories, Inc) using the Dual Luciferase Reporter assay system (Promega) in accordance with the manufacturer's instructions.

### Example 9

### RNA Extraction and RT-PC

Total cellular RNA was extracted with Trizol® (Invitrogen) according to the manufacturer's instructions and first strand cDNA was synthesized using Superscript® II (Invitrogen) after DNAse treatment. cDNA were amplified using the following primers (5′-3′): human Eotaxin-3 (151 bp): (fwd): aactccg aaacaattgtactcagctg (rev): gtaactctgggaggaaacaccctctcc; human Eotaxin-2 (251 bp): (fwd): ccatagtaaccagccttc (rev): caggttcttcatgtacctc; hEotaxin-1 (425 bp) tgaagcttgggccagcttctgtcccaacc and ggtcgactggagttggagatttttggtc; GAPDH (400 bp): tggaaatcccatcaccatct and gtcttctgggtggcagtgat; IL-13 R1 (509 bp): (fwd): ggagccagctcaatttgtag (rev) cacacgggaagttaaaggca; IL-13R2 (396 bp): (fwd): taaagttcaggatatggattgcgt (rev) cctccaaatagggaaatctgc; IL-4Rα (335 bp): (fwd): gacctggagcaacccgtatc (rev): catagcacaacaggcagacg; IL-13(96 bp): (fwd): acagccctcagggagctca (rev): tcaggttgatgctccataccat IL-4(105 bp): (fwd): acatctttgctgcctccaa(rev): aggcagcgagtgtccttct and the human hydrolysis probe #83 (Roche). Amplifications were performed in an automated thermal cycler (denaturation: 96° C., 30 sec; annealing: 60° C., 30 sec; extension: 72° C., 1 min). Amplifications of 23, 25, 30, 35 cycles were performed for GAPDH, human eotaxin-3, interleukin receptor chains and human eotaxin-1 and 2, respectively. In addition to conventional PCR, real-time PCR was carried out by rapid-cycling using the LightCycler® instrument (Roche) and IQ5 (Biorad) and LightCycler® fast start DNA master SYBR® green I (Roche) or SYBR® mix (Biorad) as a ready-to-use reaction mix according to the manufacturer's instructions.

### Example 10

### Flow Cytometry Analysis

Flow cytometry analysis of interleukin receptor chains (hIL-4Rα, hIL-13Rα1 and hIL-13Rα2) was performed as described previously Blanchard, C. et al., (2005) Int. J. Biochem. Cell Biol. 37: 2559-2573. Briefly, the TE-7 cell line was incubated with 1 μg of monoclonal antibodies, anti-human IL-4Rα, anti-human IL-13Rα1 (mAb IgG1, Diaclone) or anti-hIL-13Ra2 or IgG1 isotype, in PBS 2% fetal calf serum 0.01% sodium azide for 20 min at 4° C. after washing, cells were incubated with 0.4 μg of FITC-labeled secondary antibody. Cells were then analyzed for fluorescence by single color flow cytometry using a flow cytometer FACScalibur™ and was analyzed using FlowJo software (TreesStar, Inc., Ashland, Ore.)

### Example 11

### ELISA Protocol for IL-13, Eotaxion-1-2 and -3 Quantification

Eotaxin-1, 2 and 3 Duo set ELISA were performed according to the manufacturer's instruction (R&D) and as previously described Blanchard, C. et al., (2006) J. Clin. Invest. 116: 536-547. IL-13 Quantikine® Kit (R&D) was used to quantify IL-13 protein levels. The detection limits were respectively 70, 200, 200 and 62 μg/mL for eotaxins-1, -2, -3 and IL-13, respectively. The optical density of each well was read at a wavelength of 450 nm.

### Example 12

### Results

### Eotaxin-3 Expression in Esophageal Epithelial Cell Lines Following IL-13 Stimulation

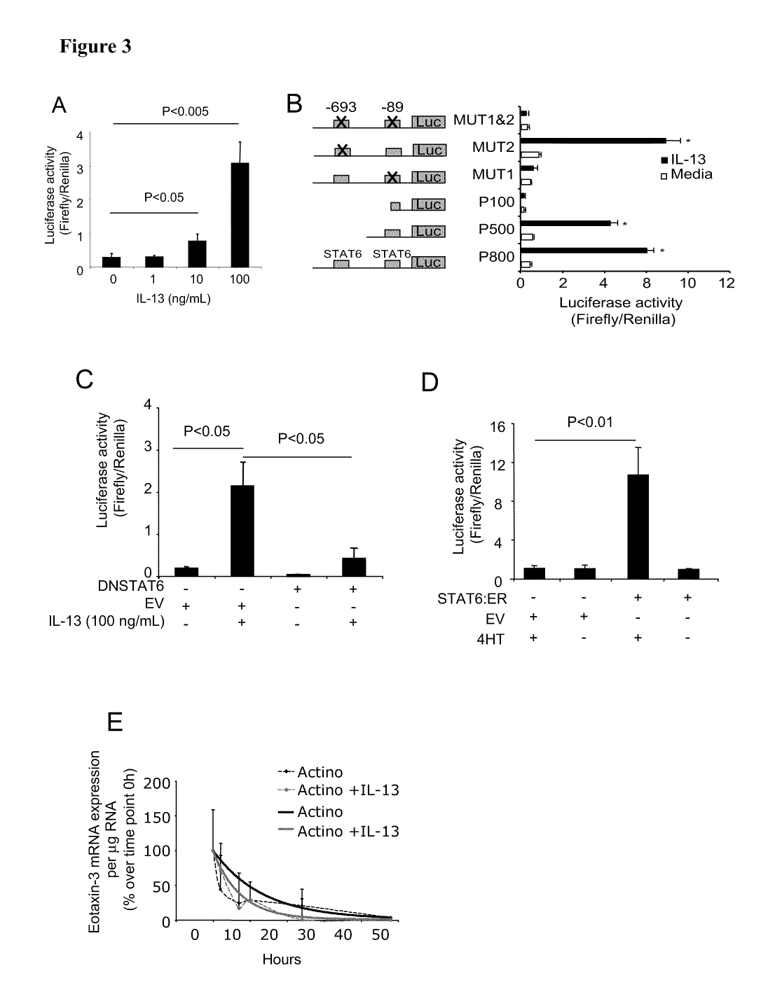

To investigate the molecular mechanisms involved in IL-13-induced eotaxin-3 expression in keratinocytes, human esophageal epithelial cell lines (TE-1, TE-6, TE-7, and TE-13) were examined. First, RT-PCR results demonstrated that the receptor chains of IL-13 (IL-13Rα1, IL-13Ra2, and IL4Rα) are expressed by these cell lines (FIG. 3A). The respective protein products were detected by means of FACS analysis in TE-7 esophageal epithelial cells (FIG. 3B). All cell lines were subsequently stimulated with increasing concentrations of human IL-13 for 0 to 48 hours (FIG. 3C). Eotaxin-3 mRNA expression was increased in a dose-dependent manner, although to varying degrees between the cell lines. Eotaxin-3 mRNA expression was increased by 4-, 8-, 77-, and 1007-fold in TE-1, TE-6, TE-7, and TE-13 cells, respectively, after IL-13 treatment at 100 ng/mL. Baseline eotaxin-3 protein expression was less than the detection limit of 200 pg/mL but was overexpressed in the cell contents and in the supernatant of IL-13-stimulated TE-7 cells in a dose- and time-dependent fashion (FIGS. 3D and E). After IL-13 stimulation (100 ng/mL for 24 hours), 2.5+/−0.7 μg/mL and 0.55+/−0.37 μg/mL eotaxin-3 was released in the supernatants of TE-7 and TE-13 cells, respectively. Notably, eotaxin-1 and eotaxin-2 expression levels were less than or at the detection limit of the real-time PCR and were not overexpressed in these cell lines after IL-13 stimulation.

### Example 13

### Results

### Characterization of the Eotaxin-3 5′UTR

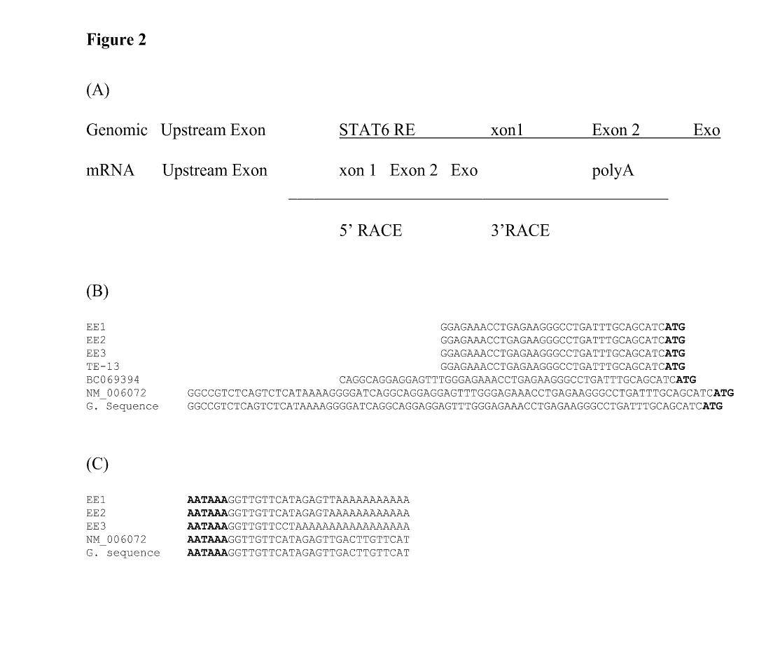

The research described herein aimed to further uncover the mechanism by which IL-13 induced eotaxin-3 in esophageal keratinocytes, focusing on transcriptional regulation. Preliminary studies aimed to characterize the 5′UTR of the eotaxin-3 gene in order to define putative promoter elements that are relevant in esophageal keratinocytes. Notably, the 5′UTR of eotaxin-3 has not been clearly established; several sequences have been published that define distinct transcriptional start sites. Moreover computational analyses have shown that an additional 5′ upstream exon is present in the eotaxin-3 gene Clark, H. F. et al., (2003) Genome Res. 13: 2265-2270. Thus, 5′ RACE analysis was performed to uncover the putative promoter region that governs increased eotaxin-3 gene transcription in EE patients and in the esophageal epithelial cell lines following IL-13 stimulation (FIG. 2). In both cases, the exact same 5′UTR was detected, composed of 34 nucleotides directly upstream of the ATG site in the genomic DNA. As such, these results establish that the 5′UTR is encoded by the same exon as the ATG start site (FIG. 2), thereby identifying the immediate 5′ region as the putative promoter in esophageal epithelial cells. These results also demonstrate that the additional first exon discovered by in silico analysis by Clark H. F. et al., (2003) Genome Res. 13: 2265-2270 is unlikely to be part of the eotaxin-3 gene.

### Example 14

### Results

### Eotaxin-3 Promoter Activity

Activated STAT6 has been shown to bind to consensus sites located at −698 and -89 bp in skin and kidney fibroblasts and epithelial cell lines of the lung and colon. Blanchard, C. et al., (2005) Int. J. Biochem. Cell Biol. 37: 2559-2573; Hebenstreit, D. et al., (2005) Mol.

Immunol. 42: 295-303; Hoeck, J. and Woisetschlager, M., (2001) J. Immunol. 167: 3216-3222; Yuan, Q. et al., (2006) Eur. J. Immunol. 36: 2700-2714. To determine the STAT6 element or elements involved in the eotaxin-3 stimulation observed in esophageal cell lines, the TE-7 cell line was transiently transfected with the full-length eotaxin luciferase reporter plasmid and truncated versions (FIG. 3). There was a significant increase of 2.7+/−0.8-fold and 10.8+/−2.2-fold of luciferase activity with the full-length reporter plasmid at IL-13 doses of 10 and 100 ng/mL, respectively (FIG. 3A). Similar results were observed in the other TE cell lines (data not shown). The TE-7 cell line was transfected with truncated promoter constructs or with a full-length promoter containing specific mutations within the STAT6 binding sites to map the relevant cis-acting promoter sequences (FIG. 3B). After IL-13 stimulation, the construct P100 (containing a truncated proximal STAT6 site) had no activity, whereas the promoter containing 800 or 500 bp had full IL-13-induced activity. Furthermore, the construct containing the mutated -89 binding site had no increase in luciferase activity, demonstrating that the proximal STAT6-responsive element at base pair -89 was required for IL-13-induced eotaxin-3 promoter activity.

### Example 15

### Results

### STAT6 Dependent Mechanism Cells were first cotransfected with the eotaxin-3 reporter and a dominant-negative STAT6-expressing vector or empty control vector to definitively implicate STAT6 in eotaxin-3 induction (FIG. 3C). The overexpression of the dominant-negative STAT6 dramatically decreased (P<0.05) activity of the IL-13-induced eotaxin-3 promoter. The cells were also cotransfected with the eotaxin-3 promoter construct and an estrogen-inducible form of STAT6 (fusion between STAT6 and estrogen receptor “ER”, STAT6:ER. Kamogawa, Y. et al., (1998) J. Immunol. 161: 1074-1077. Subsequently, addition of 4-hydroxytamoxifene to the culture medium (which allows dimerization of the modified ER protein fused to STAT6. Pritchard, C. A. et al., (1995) Mol. Cell Biol. 15: 6430-6442 resulted in a significant (P<0.01) induction of the eotaxin-3 promoter luciferase activity within 24 hours (FIG. 3E).

### Example 16

### Results

### Eotaxin-3 mRNA Stability

Chemokine mRNA expression can be highly dependent on post-transcriptional mechanisms such as RNA stability. In vitro studies have convincingly demonstrated that chemokine 3′UTR sequences are involved in promoting mRNA stability. Atasoy, U. et al., (2003) J. Immunol. 171: 4369-4378. To test if the 3′UTR of eotaxin-3 mediates mRNA stabilization upon IL-13 stimulation, first the 3′ UTR region of the eotaxin-3 mRNA in esophageal epithelial cells was defined. 3′ RACE (FIG. 2C) revealed that the polyadenylation signal was positioned 142 bp downstream from the stop codon consistent with published findings. Kitaura, M. et al., (1999) J. Biol. Chem. 274: 27975-27980. Polyadenylation occurred 10-16 bp downstream from the polyadenylation signal sequence AATAAA (FIG. 2C). Experiments using the RNA polymerase II inhibitor actinomycin-D revealed that the stability of eotaxin-3 mRNA was not significantly different between IL-13 treated and non-treated TE-7 cells (FIG. 3F). The area under the curves (AUC) were not significantly different in the presence or absence of IL-13 (p=0.8) and the half-life of eotaxin-3 in esophageal keratinocytes was determined to be 5.7±2.3 hours and not significantly modified by IL-13 treatment (FIG. 3F). Additionally, the eotaxin-3 3′UTR sequences were subcloned downstream of a SV40 promoter driven luciferase gene in the pGL3 reporter plasmid. Transfections were performed in the presence or absence of IL-13. A statistically significant increase of luciferase activity following IL-13 stimulation was not shown (data not shown).

### Example 17

### Results

### IL-13-Induced Eotaxin-3 Expression Ex Vivo

Primary keratinocytes were stimulated with 0 to 100 ng/mL human IL-13 for 48 hours to further test whether IL-13 induces eotaxin-3 mRNA expression in the esophagi of patients with EE. Eotaxin expression was studied by means of real-time PCR (FIG. 4A). Eotaxin-3 expression was induced in a dose-dependent manner after IL-13 stimulation. Remarkably, there was a 1000- and 10.000-fold increase of eotaxin-3 mRNA expression 48 hours after 10 and 100 ng/mL IL-13, respectively. Although not detectable at baseline, IL-13 (100 ng/mL) induced a dramatic release of eotaxin-3 protein (FIG. 4B) into the supernatant (6.03±0.8 ng/mL), representing at least a 30-fold increase over the detection limit (200 pg/mL). Eotaxin-1 and eotaxin-2 mRNA and protein were not overexpressed under these conditions (data not shown).

### Example 18

### Results

### IL-13-Induced Esophageal Epithelial Genes

Primary esophageal epithelial cells from the esophagi of patients with EE were cultured and stimulated with 100 ng/mL IL-13 for 48 hours to identify IL-13-induced esophageal epithelial genes. The mRNA was subjected to global transcript-expression profile analysis and normalized pair wise to unstimulated controls. Genes were filtered to fold change in biopsy specimens from patients with EE compared with those from healthy patients and IL-13 stimulated epithelial cells. A highly significant positive correlation (P<0.0001, Spearman) between modified genes in biopsy specimens from patients with EE and in IL-13-stimulated keratinocytes was observed (FIG. 4C). Eotaxin-3, cadherin-26, and TNF-α-induced protein 6 were upregulated in both specimens from patients with EE and IL-13-stimulated keratinocytes. Mast cell genes (carboxypeptidase A3), eosinophil genes (Charcot Leyden Crystal protein) and lymphocyte genes (immunoglobulin chains) were upregulated more than 10-fold in patients with EE compared with values in healthy patients. Blanchard, C. et al., (2006) J. Clin. Invest. 116: 536-547 but were not upregulated in IL-13-stimulated epithelial cells (FIG. 4C). In IL-13 stimulated cells, 4698 and 952 genes were significantly modified by using different stringencies of statistical comparisons (P<0.05 and P<0.01, respectively) compared with unstimulated cells. There were 1333 genes modified by more than 1.5-fold (780 upregulated and 553 downregulated) and 371 genes modified by 2-fold or greater (255 genes were upregulated and 116 were downregulated; FIGS. 4D and 4E; Table E1 in the Online Repository at hyper text transfer protocol www<dot>jacionline<dot>org). Ontological analysis of the 780 upregulated genes revealed that the most significant biologic processes involved pathways that regulated cell cycle (P=7.04×10⁶), response to external stimulus (P=1.33×10⁻⁵), response to wounding (P=1.77×10⁻⁵), and cell proliferation (P=2.90×10⁻⁵). In contrast, downregulated genes were involved in ectoderm development (P=3.01×10⁻⁷), epidermis development (P=7.28×10⁻⁷), tissue development (P=2.53×10⁻⁵), and keratinization (P=2.63×10⁻⁵). Genes that were upregulated 5-fold or greater and downregulated 4-fold or greater in primary esophageal keratinocytes stimulated with IL-13 (100 ng/mL, 48 hours) are shown in FIG. 4D.

In the interest of determining whether the IL-13 regulated gene signature overlapped with the EE transcriptome, the IL-13 induced gene transcript profile was compared with the EE-specific transcriptome (FIGS. 4C & 4E). Notably, 126 of the IL-13-induced genes (22% of the EE transcriptome, P<0.05) overlapped with the EE transcriptome. Interestingly, the number one gene overexpressed in IL-13-stimulated keratinocytes was eotaxin-3, which was upregulated 279-fold (FIG. 4D). Remarkably, eotaxin-1 and eotaxin-2 were not overexpressed in IL-13-treated primary keratinocyte cultures. Among the genes that overlap with EE, cadherin-26 was highly upregulated (50-fold in stimulated in keratinocytes and 26-fold in EE); TNFAIP6 was also highly overexpressed in EE and IL-1 3-stimulated keratinocytes. Finally, the skin differentiation marker gene involucrin was downregulated by 4.8-fold in IL-13-stimulated cells and by 1.7-fold in EE.

### Example 19

### Results

### IL-13 Expression in Patients with Esophageal Epithelial

To establish the participation of IL-13 (and/or IL-4) in EE pathogenesis, the research aimed first to establish whether IL-13 was overproduced in the esophageal tissue of patients with EE. By using real-time PCR analysis, there was a 16-fold increase in IL-13 mRNA in patients with EE compared with that seen in healthy patients (defined as individuals with no gastrointestinal pathology) (FIG. 5A). The IL-4 mRNA level was not significantly increased in patients with EE compared with that seen in healthy patients, although a subgroup of patients showed an increase in IL-4 levels (FIG. 5B). There was a correlation between IL-13 mRNA and eotaxin-3 expression (r²=0.49; P<0.05). Furthermore, semiquantitative PCR and real-time PCR uncovered an increase in IL-13R2 chain expression in EE patients (FIG. 5C). IL-13R1 and IL-4R mRNAs were expressed but not significantly overexpressed in EE patient biopsies (data not shown). Taken together, IL-13 is overexpressed in esophageal biopsies and appropriate IL-13 receptors for IL-13 are indeed constitutively expressed in the esophagus and modified in diseased patients.

### Example 20

### Results

### Identification of Glucocorticoid Reversible Genes in EE Patients

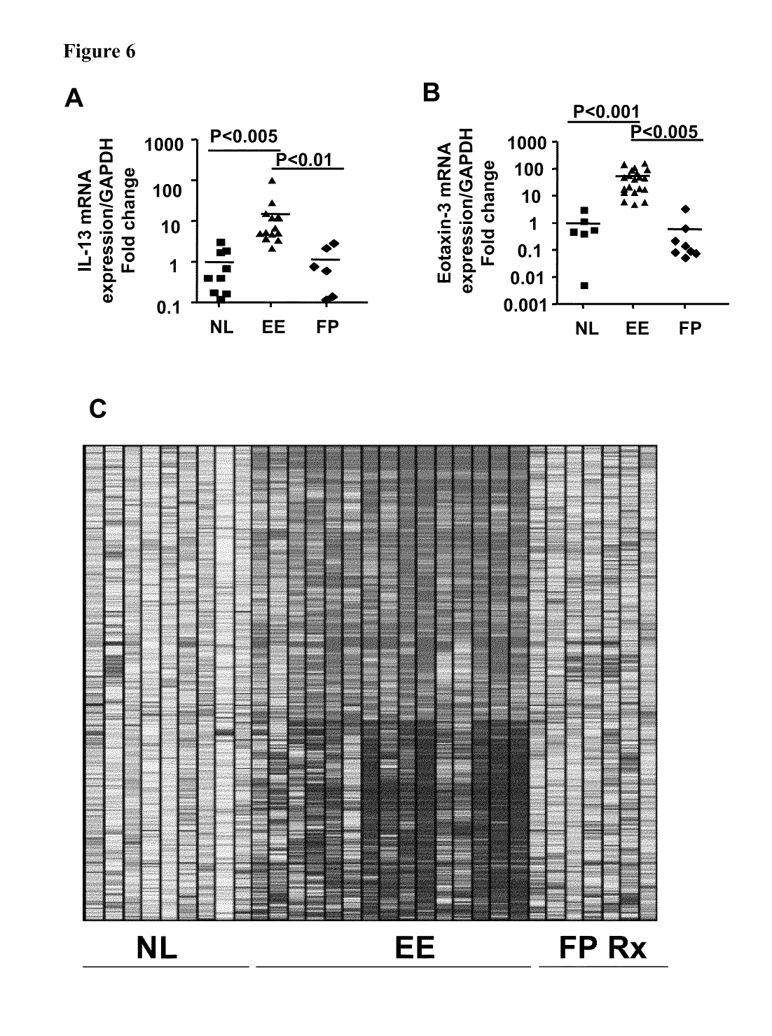

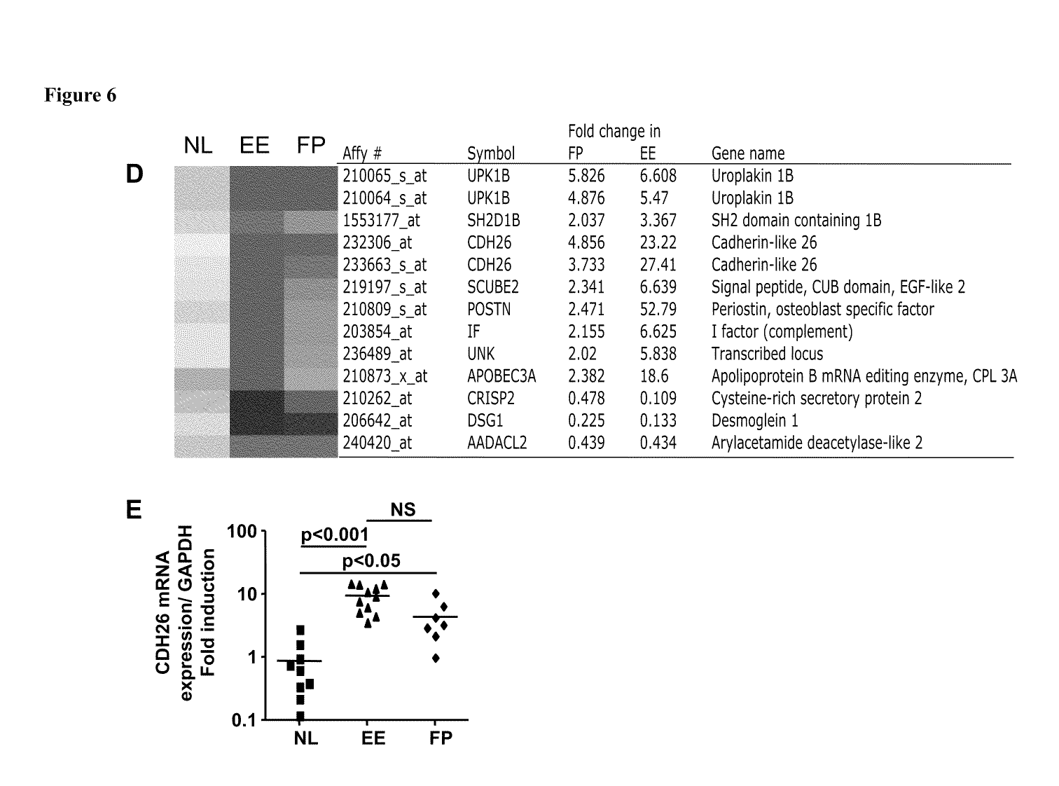

FP induces EE disease remission compared with placebo treatment. Wood, N. et al., (2003) J. Exp. Med. 197: 703-709. In patients with EE with successful anti-inflammatory intervention, IL-13 overexperssion can be normalized. Indeed, IL-13 mRNA levels were significantly (P<0.01) reduced in EE responders compared with untreated specimens, and expression levels after treatment were comparable with the levels detected in control biopsy specimens (FIG. 6A). Similarly, eotaxin-3 mRNA was normalized in FP-responder patients (P<0.01; FIG. 6B). The research next aimed to determine whether the EE transcriptome was also reversible in patients with EE successfully treated with glucocorticoids. Remarkably, 98% of the EE transcriptome was reversed to expression levels detected in biopsy specimens of healthy patients (FIG. 6C). The reversible genes include cell-specific transcripts, including eosinophil, mast cell, lymphocyte, fibroblast, and epithelial genes. In addition, genes involved in cellular recruitment and cell proliferation were also reversible. Although the biopsy specimens had no abnormal microscopic features, 12 genes were still dysregulated (FIG. 6B). Among these 12 dysregulated genes, uroplakinine and cadherin-26 remained upregulated, and desmoglein remained downregulated. Indeed, real-time PCR demonstrated a residual upregulation of cadherin-26 in FP-treated patients with EE (P<0.05; FIG. 6C).

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the embodiments of the invention. Other modifications that can be employed can be within the scope of the invention. Thus, by way of example, but not of limitation, alternative configurations of the embodiments of the invention can be utilized in accordance with the teachings herein. Accordingly, embodiments of the present invention are not limited to that precisely as shown and described.

1. A method for diagnosis of eosinophilic esophagitis (EE), comprising:
determining a level of Interleukin 13 (IL-13) expression; and
prognosing a responsive case of eosinophilic esophagitis based upon the level of IL-13 expression.

2. The method of claim 1, further comprising determining an expression level of at least one of Interleukin 4, Eotaxin-1, and Eotaxin-2.

3. A method of diagnosing an EE subtype comprising:
determining the level of at least one glucocorticoid-responsive transcript; and
diagnosing the EE subtype based upon the level of the transcript or transcripts.

4. The method of claim 3, wherein the EE subtype is responsive to FP treatment.

5. The method of claim 3, wherein the glucocorticoid-responsive transcript includes the expression of a gene described in FIG. 6(D) herein.

6. A method of diagnosing an EE subtype comprising:
determining the level of at least one EE transcriptome gene that is not a glucocorticoid-responsive transcript; and
diagnosing the EE subtype based upon the level of the transcript or transcripts.

7. The method of claim 5 or 6, wherein the glucocorticoid is fluticasone propionate.

8. A method of treating EE in an individual comprising:
determining the presence of a glucocorticoid-responsive gene expression profile; and
treating the individual based upon the profile.

9. The method of claim 8, wherein the glucocorticoid-responsive gene expression profile includes the expression of IL-13.

10. The method of claim 8, wherein IL-13 is expressed in peripheral blood mononuclear cells and/or esophageal epithelial cells.

11. A kit for the detection of a level of one or more genes associated with EE, comprising:
oligonucleotide probes complementary to subsequences of said one or more genes.

12. The kit of claim 11 wherein the probes are used in at least one of a gene chip or a PCR protocol.

13. A method of determining effectiveness of a treatment for EE, comprising:
administering the treatment to a cell, tissue, or individual; and
analyzing the cell, tissue, or individual for presence or absence of at least one of: an IL-13 response and elevated expression of at least one EE transcriptome gene.

14. A method of determining whether a reflux patient is an EE patient, comprising:
analyzing a sample from the patient to determine a profile of EE transcriptome expression, wherein EE transcriptome expression is indicative of an EE condition.


SEQUENCE LISTING (42 sequences)

SEQ ID NO: 1 - DNA, 27 bp, Artificial Sequence
  Primer
cttccaatacagccacaagccccttcc

SEQ ID NO: 2 - DNA, 27 bp, Artificial Sequence
  Primer
ggaaggggcttgtggctgtattggaag

SEQ ID NO: 3 - DNA, 45 bp, Artificial Sequence
  Primer
cccaaccacaagaaggtaccggaattgttttcagggccgtctcag

SEQ ID NO: 4 - DNA, 44 bp, Artificial Sequence
  Primer
ctgagacggccctgaaaacaattccggtaccttctgtggttggg

SEQ ID NO: 5 - DNA, 11 bp, Artificial Sequence
  Primer
ttctctcggaa

SEQ ID NO: 6 - DNA, 44 bp, Artificial Sequence
  Primer
gtaacttagttcagggatccggaatattacttaatttcctctgc

SEQ ID NO: 7 - DNA, 44 bp, Artificial Sequence
  Primer
gcagaggaaattaagtaatattccggatccctgaactaagttac

SEQ ID NO: 8 - DNA, 10 bp, Artificial Sequence
  Primer
ttcactggaa

SEQ ID NO: 9 - DNA, 29 bp, Artificial Sequence
  Primer
gatctaccatgtctctgtggggtctggtc

SEQ ID NO: 10 - DNA, 25 bp, Artificial Sequence
  Primer
taccatgtctctgtggggtctggtc

SEQ ID NO: 11 - DNA, 23 bp, Artificial Sequence
  Primer
cttcatggggtaggaagtggttg

SEQ ID NO: 12 - DNA, 27 bp, Artificial Sequence
  Primer
aattcttcatggggtaggaagtggttg

SEQ ID NO: 13 - DNA, 26 bp, Artificial Sequence
  Primer
aactccgaaacaattgtactcagctg

SEQ ID NO: 14 - DNA, 27 bp, Artificial Sequence
  Primer
gtaactctgggaggaaacaccctctcc

SEQ ID NO: 15 - DNA, 18 bp, Artificial Sequence
  Primer
ccatagtaaccagccttc

SEQ ID NO: 16 - DNA, 19 bp, Artificial Sequence
  Primer
caggttcttcatgtacctc

SEQ ID NO: 17 - DNA, 29 bp, Artificial Sequence
  Primer
tgaagcttgggccagcttctgtcccaacc

SEQ ID NO: 18 - DNA, 28 bp, Artificial Sequence
  Primer
ggtcgactggagttggagatttttggtc

SEQ ID NO: 19 - DNA, 20 bp, Artificial Sequence
  Primer
tggaaatcccatcaccatct

SEQ ID NO: 20 - DNA, 20 bp, Artificial Sequence
  Primer
gtcttctgggtggcagtgat

SEQ ID NO: 21 - DNA, 20 bp, Artificial Sequence
  Primer
ggagccagctcaatttgtag

SEQ ID NO: 22 - DNA, 20 bp, Artificial Sequence
  Primer
cacacgggaagttaaaggca

SEQ ID NO: 23 - DNA, 24 bp, Artificial Sequence
  Primer
taaagttcaggatatggattgcgt

SEQ ID NO: 24 - DNA, 21 bp, Artificial Sequence
  Primer
cctccaaatagggaaatctgc

SEQ ID NO: 25 - DNA, 20 bp, Artificial Sequence
  Primer
gacctggagcaacccgtatc

SEQ ID NO: 26 - DNA, 20 bp, Artificial Sequence
  Primer
catagcacaacaggcagacg

SEQ ID NO: 27 - DNA, 19 bp, Artificial Sequence
  Primer
acagccctcagggagctca

SEQ ID NO: 28 - DNA, 22 bp, Artificial Sequence
  Primer
tcaggttgatgctccataccat

SEQ ID NO: 29 - DNA, 19 bp, Artificial Sequence
  Primer
acatctttgctgcctccaa

SEQ ID NO: 30 - DNA, 19 bp, Artificial Sequence
  Primer
aggcagcgagtgtccttct

SEQ ID NO: 31 - DNA, 37 bp, Homo Sapiens
ggagaaacctgagaagggcctgatttgcagcatcatg

SEQ ID NO: 32 - DNA, 37 bp, Homo Sapiens; identical to SEQ ID NO: 31

SEQ ID NO: 33 - DNA, 37 bp, Homo Sapiens; identical to SEQ ID NO: 31

SEQ ID NO: 34 - DNA, 37 bp, Unknown; identical to SEQ ID NO: 31
  TE-13 cell line

SEQ ID NO: 35 - DNA, 54 bp, Unknown
  BC069394
caggcaggaggagtttgggagaaacctgagaagggcctgatttgcagcatcatg

SEQ ID NO: 36 - DNA, 81 bp, Unknown
  NM_006072
ggccgtctcagtctcataaaaggggatcaggcaggaggagtttgggagaaacctgagaagggcctgatttgcagcatcatg

SEQ ID NO: 37 - DNA, 81 bp, Unknown; identical to SEQ ID NO: 36
  G. Sequence

SEQ ID NO: 38 - DNA, 33 bp, Homo Sapiens
aataaaggttgttcatagagttaaaaaaaaaaa

SEQ ID NO: 39 - DNA, 33 bp, Homo Sapiens
aataaaggttgttcatagagtaaaaaaaaaaaa

SEQ ID NO: 40 - DNA, 33 bp, Homo Sapiens
aataaaggttgttcctaaaaaaaaaaaaaaaaa

SEQ ID NO: 41 - DNA, 33 bp, Unknown
  NM_006072
aataaaggttgttcatagagttgacttgttcat

SEQ ID NO: 42 - DNA, 33 bp, Unknown; identical to SEQ ID NO: 41
  G. Sequence